(19) United States
(12) Patent Application Publication
(10) Pub. No.: US 2008/0311172 A1
(43) Pub. Date: Dec. 18, 2008

(54) Programmed-release, nanostructured biological construct

(76) Inventors: Jay N. Schapira, Beverly Hills, CA (US); Raj Makkar, Los Angeles, CA (US)

Correspondence Address: Cislo & Thomas LLP, 1333 2nd Street, Suite #500, Santa Monica, CA, 90401-4110, US

(21) Appl. No.: 12/150,329

(22) Filed: Apr. 25, 2008

Related U.S. Application Data
(60) Provisional application No. 60/926,306, filed on Apr. 25, 2007.
(60) Provisional application No. 60/931,749, filed on May 25, 2007.
(60) Provisional application No. 60/935,021, filed on Jul. 20, 2007.
(60) Provisional application No. 60/963,290, filed on Aug. 3, 2007.

(51) Int. Cl. A61F 2/04; A61K 9/00; A61K 39/395

(52) U.S. Cl. 424/423; 424/486; 424/488; 424/173.1; 977/906

(57) ABSTRACT

A biologically engineered construct comprising of a polymeric biomatrix, designed with a nanophase texture, and a therapeutic agent for the purpose of tissue regeneration and/or controlled delivery of regenerative factors and therapeutic substances after it is implanted into tissues, vessels, or luminal structures within the body. The therapeutic agent may be a therapeutic substance or a biological agent, such as antibodies, ligands, or living cells. The nanophase construct is designed to maximize lumen size, promote tissue remodeling, and ultimately make the implant more biologically compatible. The nano-textured polymeric biomatrix may comprise one or more layers containing therapeutic substances and/or beneficial biological agents for the purpose of controlled, differential substance/drug delivery into the luminal and abluminal surfaces of the vessel or lumen, and the attraction of target molecules/cells that will regenerate functional tissue. The topographic and biocompatible features of this layered biological construct provides an optimal environment for tissue regeneration along with a programmed-release, drug delivery system to improve physiological tolerance of the implant, and to maximize the cellular survival, migration, and integration within the implanted tissues.

### CROSS-REFERENCES TO RELATED APPLICATIONS

This patent application claims priority to U.S. Provisional Patent Application Nos. 60/926,306, filed Apr. 25, 2007; 60/931,749, filed May 25, 2007; and 60/935,021, filed Jul. 20, 2007; and 60/963,290, filed Aug. 3, 2007, which applications are incorporated herein by this reference thereto.

### FIELD OF THE INVENTION

The present invention relates to the use of a biologically engineered construct that will be used for tissue regeneration and controlled drug delivery after it is implanted into tissues, vessels, or luminal structures within the body.

### BACKGROUND OF THE INVENTION

Each year, millions of patients undergo the implantation of a medical device or medication delivery system into the eye, vessels, organs, bone, cartilage, flesh, ducts and/or luminal structures within the body for the treatment of various diseases and the complications associated with these diseases. The cyto-compatibility of these implants is still imperfect, however. Implantation is often accompanied by a risk of biological rejection, cellular migration, undesirable and excessive tissue healing, clot development on the device surface, or infection. This problem has limited the application of the currently available implantable materials and technology.

For example, in the field of tissue engineering, physicians and scientists have encountered numerous problems with poor osteoblast adhesion and osteointegration following bone implant surgeries. Similarly, bladder replacement implants have been problematic, as the un-seeded polymeric scaffolds used to regenerate “new” bladder tissue, while promising, have demonstrated issues with cyto-compatibility, toxicity, and infection following placement. In vascular applications, neo-intimal proliferation is a normal response following device implantation. It is comprised of smooth muscle cell proliferation and re-endothelialization of the implant. This response essentially “indigenizes” the device, but, in 25-30% of situations, smooth muscle cell proliferation becomes excessive, and results in re-stenosis of the vascular device.

Recent and partially successful strategies to minimize these undesirable physiological processes in the treatment of vascular disease include using implantable medical devices (stents) that elute various anti-thrombogenic substances. These drug-eluting stents (“DES”) were introduced in 2003 to reduce the incidence of re-stenosis and have been successful, but perhaps at the cost of increasing stent thrombosis.

DES inhibit both smooth muscle cell proliferation and re-endothelialization, a process which reduces re-stenosis but at the same time, predisposes the patient to delayed stent thrombosis. In addition, these devices often utilize polymers as drug carriers or biofilms, such as poly(1-lactic acid) (“PLA”), poly(glycolic acid) (“PGA”), poly (lactic-co-glycolic acid) (“PLGA”), polycaprolactone, poly(ether urethane), Dacron, polytetrafluorurethane, and polyurethane (“PU”). These polymers have shown some success in large arteries, bone, and dental applications, but their surface features are not optimal and are known to be thrombogenic in small diameter vessel grafts.

Existing implant designs to both improve the biocompatibility and endothelial healing demonstrate promise, but they fail to address the critical design issue of the device: the need for surface topography and matrix formation that mimics the native biological extracellular matrix. Surface features on implantable medical devices having micro-scale resolution, and not nano-scale resolution, have proven to be inadequate, and those applications that have attempted nano-topography are generally directed at texturing the non-polymeric portion of the construct, which in many cases, is not exposed. As a result, the surface topography of the currently available implantable medical devices and/or polymers does not mimic a natural environment, limit organic bio-interaction, and do not create a suitable cellular environment for tissue regeneration.

The implantation of any therapeutic medical device immediately changes the specific tissue surface topography from nano-scale to micro-scale. Because the natural surface texture of most tissues (eye, bone, neural, bladder, organ, and intimal vascular tissue) is nanoscale (up to 100 nm) in size, recent efforts have been dedicated to improving tissue regeneration by designing biocompatible devices with nano-scale surface features. It is believed that successful implantation depends on careful replication of the cells' natural physiological and topographical environment. This includes mimicry of the composition, architecture, and surface texture of the construct. It is thought that surface chemistry (such as charge, hydrophilicity, hydrophobicity, protein adsorption) and topography (such as surface area and nano-phase surface) significantly effect how and where cells attach to biomaterials.

A number of studies have demonstrated that the nanotopographic cues of biomaterials can significantly improve cellular responses and healing both directly and indirectly. This is believed to be partially due to the fact that nano-surfaces have perhaps 40% more surface area in the Z plane and are more hydrophilic in nature. The increase in surface area in a third dimension increases device-tissue adhesion. Nanophase surface properties favor protein adsorption and interaction. Proteins contained in extracellular matrices (fibronectin, laminin, vitronectin) are nano-structured (2-70 nm) and are accustomed to interacting with nanophase surfaces, thus the adsorption of these proteins will subsequently attract endothelial progenitor cells and other reconstructive factors, stimulate healing, and can better reconstitute the injured tissue.

The latest advances in the construction of biomaterials and novel classes of biodegradeable and non-biodegradable polymers have demonstrated that materials with nanoscale surface features can better support cellular responses in vascular, bone, neural, and bladder tissue applications. Novel nanophase polymers are both compliant and cyto-compatible, as they possess the key design parameter for biocompatibility; specifically, optimal topography. More specifically, results from these studies have provided the first evidence that the surface properties of nanotextured materials and polymers preferentially enhance the competitive adhesion of endothelial cells versus vascular smooth muscle cells when compared to conventional materials. Furthermore, stem cells, when combined with nanofibers placed in the rat brain, have been shown to reverse stroke-induced neural tissue damage. There also appears to be decreased macrophage, fibroblast, B-cell, and T-cell growth on nano-surfaces, making them inherently anti-inflammatory. While much of this information is based on results from in-vitro experiments and animal studies, there is great potential to extend the existing technology to implantable medical devices for permanent or semi-permanent use in human physiological systems. Furthermore, the composition and degradation of the nano-textured polymeric material can be carefully controlled to expose functional portions of the polymer, allowing for controlled substance delivery. Thus, the “programmable” nature of the device can be used for temporal, qualitative, and quantitative release of therapeutic agents to re-create the organic physiology of tissues during in-utero tissue development, organogenesis, and/or or tissue regeneration during the healing process.

In addition to the favorable surface properties provided by nano-textured materials, the biocompatibility of implanted devices can be amplified by the addition of biologically engineered “cell sheets.” The goal of engineering cell sheets is to create a functional, differentiated tissue ex-vivo that can later be transplanted into tissues and structures within the body. By seeding cells into a biodegradable scaffold, intact cell sheets, along with their deposited extra-cellular matrices can be can be harvested and transplanted into host tissues to promote regeneration (the scaffold can also be eliminated by layering the cell sheets, creating a three-dimensional, nano-textured tissue construct).

These bio-engineering techniques are crucial when applied to regenerative medicine for improved tissue reconstruction. Because the transplanted cells within biologically engineered cell sheets retain their extra-cellular matrices, they are better able to communicate, respond to environmental cues and growth factors, and ultimately differentiate into “mature” tissue once they are implanted into the body. The carefully controlled micro- and nano-environments of these tissue constructs are more likely to maintain cellular functionality after they are implanted into host tissues. Furthermore, as the cell sheet evolves (post-implantation), the degradation of the scaffold material can be carefully controlled to expose functional portions of the underlying polymer at opportune times, allowing for controlled substance delivery. These cell sheet transplantation techniques have already shown great success in optical applications for retinal regeneration. Thus the potential to extend these applications to other organs and organ systems is great.

Therefore there is still a need for implantable medical devices designed with optimal (nanophase) surface features that are both beneficial for the tissues, and well tolerated by the body.

### SUMMARY OF THE INVENTION

The goal of this unique, “programmable” invention is to provide a method and a biological construct for addressing the problem of poor biological and physiological tolerance following medical device placement by adding a nanophase surface texture to the implantable device.

The biological construct for improved, timed-release drug delivery and tissue remodeling following implantation, comprises a polymeric biomatrix, either with or without a polymeric bioscaffold having a nanophase surface texture designed to mimic the specific extracellular matrix of a tissue into which the polymer is implanted to improve the biocompatibility of the biological construct; and various therapeutic agents seeded within the polymeric biomatrix to promote positive tissue remodeling and organ function through controlled drug delivery, optimized cyto-compatible surface characteristics, favorable protein adsorption, and improved cellular interaction. The therapeutic agent may be a therapeutic substance such as a drug, chemical compound, biological compound, or a living cell.

### BRIEF DESCRIPTION OF THE DRAWINGS

FIG. 1 illustrates the formation of the biological construct of the present invention;

FIG. 2 shows a cross-section of an embodiment of the biological construct of the present invention;

FIG. 3 illustrates another embodiment of the formation of the biological construct of the present invention;

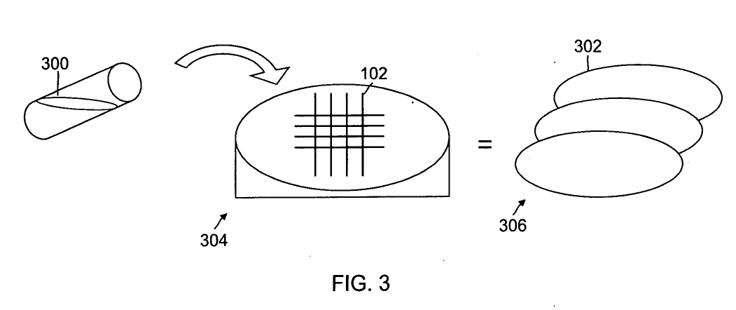

FIG. 4 shows a cross-section of another embodiment of the biological construct of the present invention;

FIG. 5 shows an embodiment of the biological construct as applied to a medical device; and

FIG. 6 shows an embodiment of the biological construct as applied to a hydrogel.

### DESCRIPTION OF THE PREFERRED EMBODIMENTS

The detailed description set forth below in connection with the appended drawings is intended as a description of presently-preferred embodiments of the invention and is not intended to represent the only forms in which the present invention may be constructed and/or utilized. The description sets forth the functions and the sequence of steps for constructing and operating the invention in connection with the illustrated embodiments. It is to be understood, however, that the same or equivalent functions and sequences may be accomplished by different embodiments that are also intended to be encompassed within the spirit and scope of the invention.

The present invention provides a biological construct and method for tissue remodeling and/or drug delivery following medical device implantation by utilizing a cyto-compatible, layered, bio-compatible polymeric biomatrix optimally constructed with a specialized surface texture of grain sizes up to 100 nm seeded with various therapeutic agents.

The biological construct may be used as an implantable device for controlled-release drug delivery and/or tissue regeneration system. The biological construct may be non-covalently or covalently layered with coatings of organic or semi-synthetic, nano-textured polymer. The nano-textured polymer may comprise pharmaceutical substances, such as growth factors, ligands, antibodies, and/or other beneficial biologically active agents for the purposes of controlled, differential substance/drug delivery into the luminal and abluminal surfaces of the tissue, and the attraction of target molecules/cells that will regenerate functional tissue and restore anatomic and physiologic integrity to the organ. The composition and construction of the polymer will facilitate the release of therapeutic agents in a temporal order that mimics the order of physiological processes that take place during natural organogenesis and tissue regeneration.

The healing process may also be augmented by the addition of a tissue-specific, biologically engineered cell sheet, which may be overlaid onto the device along with its extracellular matrix. This may include endothelial progenitor cells, adult stem cells, embryonic stem cells, endogenous cardiac-committed stem cells, and other multipotent primitive cells capable of differentiation and restoring anatomic and physiologic integrity to the organ.

The biological construct comprises a polymeric compound designed with a nanophase surface texture, and various therapeutic agents, for the purpose of tissue regeneration and/or controlled delivery of growth factors and drugs after it is implanted into tissues, vessels, or luminal structures within the body. The invention may be applied to, but is not limited to any medical implant intended for vascular, cardiac, eye, bladder, cartilage, central and peripheral nervous system, lung, liver, pancreatic, stomach, smooth and skeletal muscle, visceral, renal, reproductive, epithelial and/or connective tissue application.

The following terms, as used herein, shall have the following meanings:

The term “delivery vehicle” refers to platforms, such as medical devices or medical substances that are introduced either temporarily or permanently into a mammal for the purposes of treating a disease, complication of a disease, or medical condition. This delivery vehicle can be introduced surgically, percutaneously, or subcutaneously into vessels, organs, cartilage, neural tissue, flesh, ducts and/or luminal structures within the body. Medical devices include, but are not limited to a stent, vascular graft, synthetic graft, valve, catheter, filter, clip, port, pacemaker, pacemaker lead, occluder, defibrillator, shunt, drain, clamp, probe, screw, nail, staple, laminar sheet, mesh, suture, chest tube, insert, or any device meant for therapeutic purposes. These devices may comprise titanium, titanium oxide, titanium alloy, stainless steel, nickel-titanium alloy (nitinol), cobalt-chromium alloy, magnesium alloy, carbon, carbon fiber, and/or any other biocompatible metal, alloy, or material. Medical substances include gels, such as hydrogels.

The term “nano-phase” or “nano-textured” are defined as having a surface texture with a grain size up to approximately 100 nanometers (nm). This includes, but is not limited to random or non-random patterns, which may include nano-spheres, nano-fibers, or nano-tubes.

The term “polymer” refers to when a molecule formed from the union of multiple (two or more) monomers. The polymer may be preferably amphipathic, and may be organic, semi-synthetic, or synthetic. Examples of polymers relevant to the present invention include, but are not limited to biologically tolerated and pharmaceutically acceptable poly(1-lactic acid) (“PLA”), poly(glycolic acid) (“PGA”), poly(lactic-co-glycolic acid) (“PLGA”), polycaprolactone (“PCL”), poly(ether urethane), Dacron, polytetrafluorurethane, polyurethane (“PU”), and/or silicon. The polymer may also include naturally occurring materials such as collagen I, collagen III, fibronectin, fibrin, laminin, cellulose ester, or elastin.

The term “nano polymer” or “nano-textured polymer” refers to the polymer (described above) with a naonophase surface roughness (grain size up to approximately 100 nm).

The term “therapeutic agent,” refers to any therapeutic substance or biological agent, or “beneficial biologically active agents” that is administered to the tissues or organs. of a mammal to produce a beneficial effect. With respect to the present invention, therapeutic substances include antiproliforative agents, growth factors, antibiotics, thrombin inhibitors, immunosuppressive agents, antioxidants, peptides, proteins, lipids, enzymes, vasodilators, anti-neoplasties, anti-inflammatory agents, ligands (peptides or small molecule that binds a surface molecule on target cell), linker molecules, antibodies, and any janus kinase and signal transduction and activator of transcription (“JAK/STAT”) or AKT pathway activators are especially relevant. Biological agents include adult and/or embryonic stem cells, endogenous stem cells (e.g. endogenous cardiac-committed stem cells), and progenitor cells. These therapeutic agents are meant to be seeded into the polymeric material listed above.

The term “bioscaffold” refers to the polymeric backbone or lattice where therapeutic agents may be seeded. The bioscaffold may be biodegradeable (erodable) or non-biodegradeable (depending on the application) and can made from the polymeric mediums described above, insuring that that when implanted into the body, the polymer does not produce an adverse effect or rejection of the material. The architecture of the bioscaffold will attempt to mimic the native biological extracellular matrix of the tissue it is meant to regenerate. For example, the bioscaffold surface may contain weaves, struts, and coils.

The term “biomatrix” refers to the nano-textured biological construct, with or without a bioscaffold, and the therapeutic agent seeded within (drugs, living cells, etc).

The term “biodegradeable” refers to a material that can be broken down or eroded by chemical (pH, hydrolysis, enzymatic action) and/or physical processes once implanted into the body and exposed to the in-vivo physiological environment. The kinetics of this process can take from minutes to years. The subsequent components are non-toxic and excretable.

The term “cell sheet” refers to a specialized, tissue-specific population of cells grown on a scaffold. The sheets are cultured ex-vivo and subsequently harvested, along with their extra-cellular matrices, overlaid onto the nano-textured construct, and transplanted into host tissues to promote regeneration.

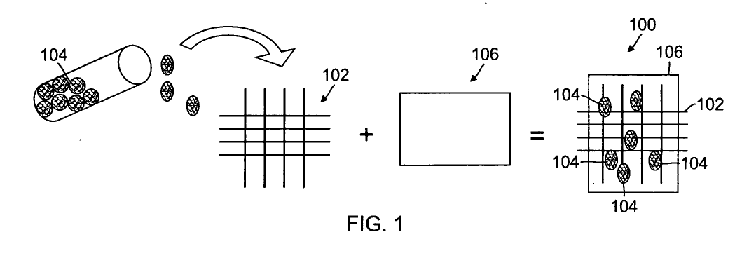

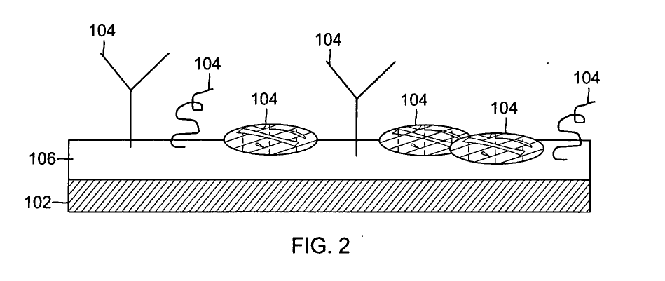

As illustrated in FIGS. 1 and 2, the nano-textured polymeric biomatrix 100 comprises an amphipathic organic, synthetic, or semi-synthetic polymeric material or bioscaffold 102 and the therapeutic agent 104 and/or 300 seeded within. The therapeutic agent 104 and/or 300 may be incorporated directly into a polymeric solution in a random or nonrandom fashion. The therapeutic agent 104 and/or 300 may be added directly, or the therapeutic agent 104 and/or 300 may be encapsulated, for example, enveloped into a microbubble, microsphere, or something of the kind before being added to the polymeric solution. The therapeutic agent 104 and/or 300 may be covalently or non-covalently coupled to the polymer. Depending on the chemical nature and molecular weight of the therapeutic agent 104 and/or 300, it may also be positioned between layers of polymers 102. The amount, concentration, or dosage of the therapeutic agent 104 and/or 300 seeded within the polmeric biomatrix 100 will be optimized for the target tissue and defined as the amount necessary to produce a therapeutic effect.

The nano-textured polymeric biomatrix 100 serves as a timed-release drug delivery system. After implantation, the construct is exposed to a physiological environment, and subsequently begins to erode and release at least one therapeutic substance 104. The erosion kinetics of the polymeric biomatrix 100 depends on the polymer density, choice of lipid membrane, glass transition temperature, and the molecular weight of the seeded substances and biological agents. In some embodiments, the biomatrix 100 may be comprised of different types and densities of polymer, so that the erosion kinetics will be different throughout the construct. This will ensure healthy tissue regeneration (via the release of therapeutic substances) along with timed substance delivery (due to the degradation of the polymer) to maximize the biocompatibility of the implantable construct. The biological construct may be constructed such that the programmable nature of the device can be used for temporal, qualitative, and quantitative release of tissue-specific, therapeutic substances. The order, type, and dosage of substances released mimics the order that is observed in naturally occurring physiological environments during in-utero tissue generation, organogenesis, and/or tissue and/or organ regeneration during healing.

Thus, the nano-textured polymeric biomatrix 100 may be designed to facilitate controlled three-dimensional drug delivery and optimized to improve tissue regeneration. For example, the polymer 102 can serve to protect or preserve the biological agents 300, as they may not be exposed to the physiological environment until the polymeric portion of the biomatrix effectively erodes. In some embodiments, the polymeric portion may be in liquid or lyophilized phase at room temperature (approximately 25° C.) and subsequently change phase or conformation after implantation or direct injection at core body temperature (approximately 37° C.).

The polymer 102 may also prepare the cellular environment by releasing buffers, inhibitors, or growth factors that will enhance the efficacy of a seeded therapeutic biological agent 300 or therapeutic substance 104 before it is released. This may also serve to protect the tissue from the acidity generated as a result of polymeric degradation.

In some embodiments, the constitution of the polymer may differ on different aspects of the construct. The surface of the polymeric bioscaffold 102 will be nano-textured to increase favorable cellular responses by optimizing surface chemistry, hydrophilicity, charge, topography, roughness, and energy. The surface of the polymeric bioscaffold 102 can be nano-textured 106 by methods described previously by Webster, et al. (5, 6, 14-18, 25, 26, U.S. patent application Ser. No. 10/793,721). Briefly, nano-textures may be generated with nanoparticles having grain sizes up to approximately 100 nm (carbon nano-tubules, helical rosette nano-tubes, nano-spheres, nano-fibers, etc). The nanoparticles may be transferred to the surface of a polymeric bioscaffold 102 comprising, for example, PLGA, PU, or the like, using specialty molds, hydrogel scaffolds, NaOH treatment, and sonication power. The surface roughness can be evaluated prior to implantation using scanning electron microscopy, if necessary. The nano-texture of each polymeric layer will not only improve the biocompatibility and cellular responses to the surface, but will also augment the bond between layers as well.

As shown in FIGS. 1 and 2, in some embodiments, a specialized population of tissue-specific cells 300 including, but not limited to, stem cells and progenitor cells, may be seeded withinin the polymeric bio-scaffold.

The nano-textured polymeric biomatrix 100 can be securely affixed to a delivery vehicle or a medical platform 400 by dipping, ultrasonic spray coating, painting, or syringe application. Dipping is a common method, and involves submerging the platform into a liquid solution (dissolved polymer) of the biomatrix. This can also be achieved by spraying the platform 400 with the liquid solution. The platform 400 can be dried and re-dipped or re-sprayed with different solutions to create specific, successive, biomatrix layers with independent functions. The multiple layers can also provide structural support for the construct and the polymeric density can be carefully controlled and altered to control elution kinetics. In addition, the concentration and combination of substances can be varied depending upon the polymeric thickness and/or number of layers in the polymer to control elution kinetics.

Select biological agents (antibodies, cells, etc.) may be covalently or non-covalently attached to the construct layers after it is dipped or sprayed. In some embodiments, the polymeric biomatrix 100 may not require a medical platform 400. Instead, it may be comprised of layers of biological agents and substances 306 with the layering providing the structural integrity.

In some embodiments, the biological construct for tissue regeneration in the present invention capitalizes on its likeness to natural architecture, nano-phase surface topography and the unique drug delivery system to improve the biocompatibility of the implantable construct 402 by attracting endothelial progenitor cells and other reconstructive factors, stimulating healing, and can better reconstituting the injured tissue.

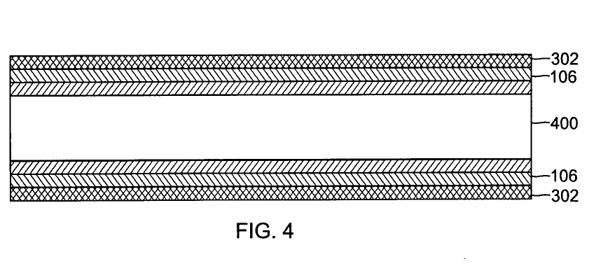

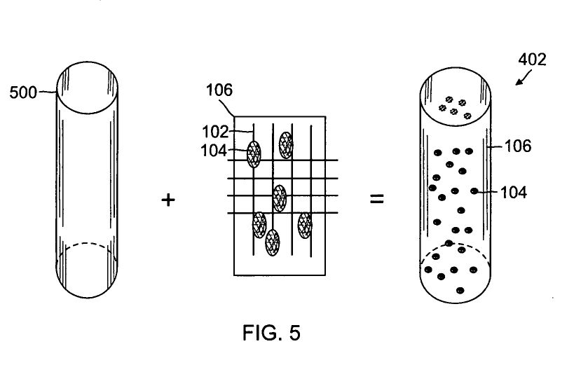

As shown in FIGS. 4-5, in some embodiments, the current invention will provide a method for addressing the problem of re-stenosis and late thrombosis following endovascular or endoluminal device placement by implanting a biological construct (polymer or polymer+platform) whose nano-surface features and polymeric constitution may enhance endothelial healing, mitigate smooth muscle vascular cell adhesion, and ultimately promote vascular reconstitution in patients suffering from cardiovascular disease. The nano-textured, polymeric biomatrix 100 can be formulated and applied (sprayed, dipped, painted) onto a device 500, such as a stent, vascular graft, valve, catheter, filter, clip, port, pacemaker, pacemaker lead, defibrillator, shunt, or any endovascular or endoluminal device designed to treat the complications associated with vascular disease. In this instance, the construct would seek to emphasize the method of endothelial healing facilitated by the nano-phase texture of the polymer and the platform of the device 500. The pattern of this nano-texture may be random and/or non-random; designed to effect the flow of blood, such as to facilitate the capture of endothelial progenitor cells; maximize lumen size; and minimize smooth muscle cell adhesion.

The polymer 102 facilitates the controlled release of pharmaceutical compound 104 to abluminal and luminal surfaces of the construct. To facilitate controlled release the biomatrix 100 may contain layers of ligands, antibodies, and growth factors designed to bind and/or attract specific membrane molecules on target cells (endothelial progenitor cells), with the goal of augmenting endothelial healing. In this embodiment, these may include one or more of the following: anti-proliferative agents (paclitaxel, sirolimus, etc.), endothelial progenitor cells, endogenous cardiac-committed stem cells, Flkl+progenitors, cardiosphere daughter cells, endothelial cell growth factors granulocyte macrophage colony-stimulating factor (“GM-CSF”, CSF-1), granulocyte colony-stimulating factor (“G-CSF”), macrophage colony-stimulating factor (“M-CSF”), erythropoietin, stem cell factor, vascular endothelial growth factor (“VEGF”), fibroblast growth factors (“FGF”) such as FGF-3, FGF-4, FGF-5, FGF-6, FGF-7, FGF-8, and FGF-9, basic fibroblast growth factor, platelet-induced growth factor, transforming growth factor beta-1, acidic fibroblast growth factor, osteonectin, angiopoetin-1, angiopoetin-2, insulin-like growth factor), smooth muscle cell growth inhibitors, antibiotics, thrombin inhibitors, immunosuppressive agents, antioxidants, peptides, proteins, growth factor agonists, vasodilators, anti-platelet aggregation agents, collagen synthesis inhibitors, extracellular matrix components, fms-like tyrosine kinase receptor-3 (“flt3”) ligand, c-mpl ligand, megakaryocyte growth and differentiation factor (“MGDF”) or thrombopoietin (“TPO”), ricin ligands, or any antibody or antibody fragment that has the binding affinity to one of the following: CD34 receptors, CD133 receptors, CDw90 receptors, CD117 receptors, HLA-DR, Flkl, VEGFR-1, VEGFR-2, Muc-18 (CD146), CD 130, stem cell antigen (Sca-1), stem cell factor (SCF/c-kit ligand), Tie-2, and/or HAD-DR. Together, this nano-textured device will promote endothelial healing and vascular reconstruction.

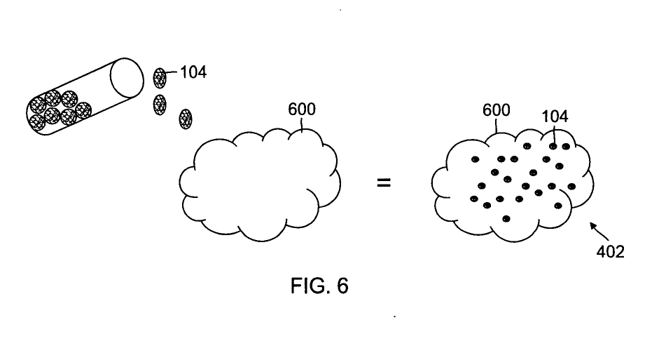

In another embodiment, the current invention provides a method for addressing the problem of cellular migration and survival following various forms of “cell therapy.” Therapeutic substances and biologically beneficial agents 104 and 300, respectively, can be applied directly to a specific lesion or insult in the tissue through the use of a nano-textured hydrogel 600 seeded with therapeutic agents 104 and/or 300 as shown in FIG. 6. Using minimally invasive surgical techniques to apply the gel 600, or “bio-dots,” the use of this polymeric medium can ensure proper placement and security of the cells, discourage cellular migration, improve cellular response, survival, and integration, and protect protein based substances seeded within. Additionally, the elution kinetics of the construct can be controlled by the rate of polymeric degradation, making the “bio-dots” inherently programmable.

In another embodiment, the current invention provides a method for addressing the problem of cellular rejection, migration, and partial thrombosis of the hepatic vasculature following islet transplantation procedures in insulin-dependant diabetic patients. Type I and late stage type II diabetics have impaired insulin and glucagon function, which compromises their endogenous ability to maintain euglycemia. In attempting to manage blood glucose levels, most patients undergo rigorous insulin replacement therapy in the form of subcutaneous insulin administration. While there have been advances in glycemic monitoring devices and insulin delivery systems, insulin therapy is still flawed; it is unable to mimic physiological insulin secretion, making patients extremely vulnerable to complications, primarily hypoglycemia. In an attempt to mitigate these complications, and to free patients of insulin dependency, experimental islet transplantation has become an option.

As with many transplantations, this procedure is accompanied by the risk of partial thrombosis in the portal vein (and other small intra-hepatic vessels), islet cell rejection, poor cellular survival and function, and cellular migration. Additionally, anti-rejection drugs (immuno-suppressants) given after transplantation make patients vulnerable to opportunistic infection and have been shown to impair normal islet function. By seeding the islets 300 in a nano-textured polymeric bioscaffold 102, 106, the islets will be carefully deposited along with their extra-cellular matrices and growth factors through the portal vein into the hepatic host tissue. The polymer 102 will provide a stable, therapeutic environment for the islets, which will bio-mimic physiological conditions and encourage proper function. The ultimate goal of this application is to stimulate integration, and ultimately improve overall insulin and glucagon secretion. Functional islets may free diabetic patients of insulin dependency or reduce insulin dependency and allow them to realize the benefits of true glycemic control.

The nanophase surface properties of the construct will favor positive tissue remodeling following implantation through controlled drug delivery, optimized cyto-compatible surface characteristics, and favorable protein adsorption and cellular interaction. The application of the present invention may extend to, but is not limited to biological constructs in vascular, cardiac, epithelial, eye, bladder, cartilage, central and peripheral nervous system, lung, liver, pancreatic, stomach, smooth and skeletal muscle, visceral, renal, reproductive, and connective tissues.

While the current invention is unique compared to previous developments in the field, it seeks to emphasize the improved biocompatibility of the device, the controlled drug delivery system, and the nanophase surface features of the polymer.

The foregoing description of the preferred embodiment of the invention has been presented for the purposes of illustration and description. It is not intended to be exhaustive or to limit the invention to the precise form disclosed. Many modifications and variations are possible in light of the above teaching. It is intended that the scope of the invention not be limited by this detailed description, but by the claims and the equivalents to the claims appended hereto.

What is claimed is:

1. A biological construct for improved drug delivery and tissue remodeling, comprising:
a. a polymeric biomatrix, comprising:
i. a biocompatible polymer having a nanophase surface texture designed to mimic the specific extracellular matrix of a tissue into which the polymeric biomatrix is implanted to improve the biocompatibility of the biological construct; and
ii. therapeutic agents seeded within the biocompatible polymer.

2. The biological construct of claim 1, wherein the biocompatible polymer is selected from the group consisting of an organic material, a synthetic material, and a semi-synthetic material.

3. The biological construct of claim 2, wherein the biocompatible polymer is selected from the group consisting of poly(1-lactic acid) (“PLA”), poly(glycolic acid) (“PGA”), poly(lactic-co-glycolic acid) (“PLGA”), polyethylene glycol (“PEG”), polycaprolactone (“PCL”), poly (N-isopropylacrilamide) (“PIPAAm”), poly(ether urethane), Dacron, polytetrafluorurethane, polyurethane (“PU”), cellulose ester, collagen I, collagen III, elastin, fibronectin, fibrin, fibrinogen, laminin, and silicon

4. The biological construct of claim 1, wherein the nanophase surface texture comprises nanoparticles selected from the group consisting of nano-tubules, nano-fibers, and nano-spheres.

5. The biological construct of claim 4, wherein the nanophase surface texture has a grain size up to approximately 100 nanometers.

6. The biological construct of claim 4, wherein the nanoparticles are arranged in a predetermined pattern.

7. The biological construct of claim 4, further comprising a plurality of nanophase surface texture regions, wherein each nanophase surface texture region has a pattern independent of another nanophase surface texture region.

8. The biological construct of claim 1, wherein the therapeutic agent is selected from the group consisting of a ligand, an antibody, a growth factor, an anti-proliferative agent, an adult stem cell, an embryonic stem cell, an endogenous cardiac-committed stem cell, an endothelial progenitor cell, an endothelial cell growth factor, granulocyte macrophage colony-stimulating factor (“GM-CSF”), granulocyte colony-stimulating stimulating factor (“G-CSF”), macrophage colony-stimulating factor (“M-CSF”), erythropoietin, a stem cell factor, vascular endothelial growth factor (“VEGF”), a janus kinase and signal transduction and activator of transcription anti-inflammatory agent pathway activator (“JAK/STAT”), an AKT/Pim-1 pathway activator, an AKT/Pim-3 pathway activator, thymosin beta-4, FGF-3, FGF-4, FGF-5, FGF-6, FGF-7, FGF-8, FGF-9, a basic fibroblast growth factor, a platelet-induced growth factor, transforming growth factor beta-1, an acidic fibroblast growth factor, osteonectin, angiopoetin-1, angiopoetin-2, an insulin-like growth factor, a smooth muscle cell growth inhibitor, an antibiotic, a thrombin inhibitor, an immunosuppressive agent, an antioxidant, a peptide, a protein, a growth factor agonist, a linker molecule, a vasodilator, an anti-platelet aggregation agent, a collagen synthesis inhibitor, an extracellular matrix component, flt3 ligand, c-mpl ligand, a ricin ligand, a buffer, and an enzyme.

9. The biological construct of claim 8, wherein the agent is an antibody that has an affinity to a receptor selected from the group consisting of CD34 receptors, CD 133 receptors, CDw90 receptors, CD117 receptors, HLA-DR, Flkl, VEGFR-1, VEGFR-2, Muc-18 (CD146), CD 130, stem cell antigen (Sca-1), stem cell factor (SCF/c-kit ligand), Tie-2, and HAD-DR.

10. The biological construct of claim 1, comprising a plurality of polymeric biomatrices arranged in layers for careful coordinated execution of drug release from the polymer.

11. The biological construct of claim 10, wherein each layer comprises an independent therapeutic agent.

12. The biological construct of claim 1, further comprising a delivery vehicle selected from the group consisting of a device and a gel.

13. The biological construct of claim 12, wherein the delivery vehicle is a device selected from the group consisting of a stent, a vascular graft, a synthetic graft, a valve, a catheter, a filter, a clip, a port, a pacemaker, a pacemaker lead, an occluder, a defibrillator, a shunt, a drain, a clamp, a probe, a screw, a nail, a staple, a laminar sheet, a mesh, a suture, a chest tube, and an insert.

14. The biological construct of claim 13, wherein the device is made of at least one metal from the group consisting of titanium, titanium oxide, titanium alloy, stainless steel, nickel-titanium alloy, cobalt-chromium alloy, magnesium alloy, carbon, carbon fiber.

15. The biological construct of claim 12, wherein the delivery vehicle is a hydrogel.

16. The biological construct of claim 1 further comprising a polymeric bioscaffold into which the therapeutic agents are seeded.

17. The biological construct of claim 1, wherein the therapeutic agent is a tissue-specific, therapeutic substance and the biological construct is programmable for a temporal, qualitative, and quantitative release of the tissue-specific, therapeutic substance.

18. The biological construct of claim 17, wherein the temporal, qualitative, and quantitative release of the tissue-specific, therapeutic substance mimics a release that is observed in a naturally occurring physiological environment during in-utero tissue generation, organogenesis, and/or organ and/or tissue regeneration during healing.

19. A method of creating a first biological construct for improving drug delivery and enhancing tissue regeneration, comprising:
a. providing a first biocompatible polymer having a nanophase surface texture designed to mimic the specific extracellular matrix of a tissue into which the first biocompatible polymer is implanted to improve the biocompatibility of the biological construct; and
b. seeding therapeutic agents within the biocompatible polymer to form a first polymeric biomatrix.

20. The method of claim 19 further comprising applying the first polymeric biomatrix to a delivery vehicle.

21. The method of claim 20, wherein the delivery vehicle is selected from the group consisting of a device or a gel.

22. The method of claim 21, wherein the delivery vehicle is a device selected from the group consisting of a stent, a vascular graft, a synthetic graft, a valve, a catheter, a filter, a clip, a port, a pacemaker, a pacemaker lead, an occluder, a defibrillator, a shunt, a drain, a clamp, a probe, a screw, a nail, a staple, a laminar sheet, a mesh, a suture, a chest tube, and an insert.

23. The method of claim 20, wherein the application step is selected from a group consisting of spraying, dipping, ultrasonic spray coating, painting, and applying with a syringe.

24. The method of claim 20, further comprising the steps of:
a. drying the polymeric biomatrix; and
b. applying a second polymeric biomatrix to create a layer of polymeric biomatrices, wherein the each polymeric biomatrix comprises an independent therapeutic agent.

25. The method of claim 19, wherein the first biocompatible polymer is created from a polymeric material selected from the group consisting of an organic compound, a synthetic compound, and a semi-synthetic compound.

26. The method of claim 19, wherein the first biocompatible polymer is created by a technique selected from the group consisting of a specialty mold, a hydrogel, and a sodium hydroxide sonication.

27. The method of claim 19, further comprising layering a second biocompatible polymer on top of the first biocompatible polymer, wherein the therapeutic agents are contained in between the first and second biocompatible polymer.

28. The biological construct of claim 19 further comprising providing a polymeric bioscaffold into which the therapeutic agents are seeded.

29. The biological construct of claim 19, wherein the therapeutic agent is a tissue-specific, therapeutic substance and the biological construct is programmable for a temporal, qualitative, and quantitative release of the tissue-specific, therapeutic substance.

30. The biological construct of claim 29, wherein the temporal, qualitative, and quantitative release of the tissue-specific, therapeutic substance mimics a release that is observed in a naturally occurring physiological environment during in-utero tissue generation, organogenesis, and/or organ and/or tissue regeneration during healing.
